(19) United States
(12) Patent Application Publication
(10) Pub. No.: US 2019/0258930 A1
(43) Pub. Date: Aug. 22, 2019

(54) APPARATUS FOR ASCERTAINING PREDICTED SUBJECTIVE REFRACTION DATA OR PREDICTED CORRECTION VALUES, AND COMPUTER PROGRAM

(71) Applicant: Carl Zeiss Vision International GmbH, Aalen (DE)

(72) Inventors: Arne Ohlendorf, Tübingen (DE); Siegfried Wahl, Donzdorf (DE); Christian Leibig, Munich (DE); Alexander Leube, Tübingen (DE)

(21) Appl. No.: 16/404,991

(22) Filed: May 7, 2019

Related U.S. Application Data
(63) Continuation of application No. PCT/EP2017/078161, filed on Nov. 3, 2017.

(30) Foreign Application Priority Data
Nov. 14, 2016 (EP) 16198607.0

(51) Int. Cl. G06N 3/08; G16H 50/20

(52) U.S. Cl. CPC G06N 3/08; G16H 50/20

(57) ABSTRACT

An apparatus for ascertaining predicted subjective refraction data or predicted subjective correction values of an eye to be examined on the basis of objective refraction data of the eye to be examined is disclosed. The apparatus includes an evaluation device with a calculation unit, which ascertains the predicted subjective refraction data or predicted subjective correction values of the eye from the objective refraction data of the eye with a function. The function is a nonlinear multidimensional function or a family of nonlinear multidimensional functions, which is the result of training a regression model or classification model, wherein the regression model or classification model has been trained on the basis of a training data record, which, for a multiplicity of subjects, in each case includes at least objective refraction data and assigned ascertained subjective refraction data or assigned ascertained subjective correction values.

### CROSS REFERENCE TO RELATED APPLICATIONS

This application is a continuation application of international application PCT/EP2017/078161, filed Nov. 3, 2017, which claims priority to European patent application EP 16198607.0, filed Nov. 14, 2016, both of which are hereby incorporated by reference in their entireties.

### TECHNICAL FIELD

The present disclosure relates to an apparatus for ascertaining predicted subjective refraction data or predicted subjective correction values for an eye on the basis of measured objective refraction data of the eye. Additionally, the disclosure relates to a computer program having program code, which facilitates the ascertainment of predicted subjective refraction data or predicted subjective correction values for an eye on the basis of measured objective refraction data of the eye.

### BACKGROUND

Refraction determination relates to determining the refractive properties of the (human) eye. In the case of refraction determination, it is possible to distinguish between subjective refraction determination and objective refraction determination.

In this case, methods for subjective refraction determination are based on (subjective) feedback from a person to be examined with respect to the person's visual perception. One example for a subjective refraction determination is a determination of the refractive properties on the basis of eye charts with ever decreasing optotypes (e.g., numbers or letters) or ever decreasing symbols, in which case the person to be examined provides feedback with respect to which characters can be discerned by the person.

Conventionally, the subjective refraction determination is implemented by means of a trial frame and trial lenses or a manual or digital phoropter and using optotypes, which are displayed on an external monitor. By way of example, phoropters are described at the url: de.wikipedia.org/wiki/Phoropter, as of Oct. 13, 2016. Here, a person to be examined observes the optotypes and an optician or ophthalmologist inserts different trial lenses with different corrective powers into the trial frame, or changes a correction setting if a phoropter is used. The person to be examined then provides feedback in respect of what trial lenses or what settings of the phoropter allow the optotypes to be recognized to the best possible extent.

Here, the trial lenses used in the process or the corresponding correction settings of the phoropter are in each case assigned to a certain refractive error, which is corrected by the respective trial lens or the setting of the phoropter. Here, the subjective refraction determination explained above can be implemented separately for each eye (in monocular fashion) or else for both eyes together (in binocular fashion).

Methods and apparatuses for objective refraction determination require no such feedback from the person to be examined with regard to the person's visual perception. The refractive errors are measured, with computational preparation where necessary, from the directly measured variables. By way of example, WO 2004/112576 A2 describes a method and an apparatus for determining a measure of visual acuity on the basis of a measured wavefront aberration. Here, the measure for visual acuity is calculated from the wavefront aberration of lower and higher order using a pointspread function. By way of example, the objective refraction determination is carried out with the aid of autorefractors, photorefractors, wavefront analyzers, retinoscopes, etc.

The above-describe procedure is substantially stationary; i.e., it is bound to the premises of the optician or ophthalmologist and requires an appropriately trained optician or ophthalmologist to carry out the refraction determination, determine refractive errors, and, where necessary, adequately correct the latter, for example by prescribing spectacles. A vivid explanation of the differences between subjective refraction, objective refraction and prescription can be gathered from the url: de.wikipedia.org/wiki/Refraktion_(Augenoptik), downloaded on Oct. 21, 2016, for example.

Data that describe the ametropia of the eye (colloquially, the refractive error of the eye), are referred to as refraction data within the scope of this description. Here, subjective refraction data are data obtained by means of subjective refraction; objective refraction data are those that were ascertained by means of objective refraction determination. The subjective refraction data and/or the objective refraction data can be specified, for example, in the form of Zernike coefficients, using the conventional notation of sphere, cylinder, and axis, where sphere denotes the spherical refractive value S, cylinder denotes the cylindrical refractive value C, and axis denotes the position of the cylinder axis (axis orientation), using the power vector notation (SÄ, J₀, J₄₅), where SÄ denotes the spherical equivalent, J₀ denotes the horizontal or vertical component of the Jackson cross cylinder, and J₄₅ denotes the components of the Jackson cross cylinder extending at an angle of 45° or 135° in relation to the horizontal or vertical component, or using any other suitable notation. The power vector components SÄ, J₀, and J₄₅ are related to the values of S, C, and α for sphere, cylinder, and axis by way of the equations:

SÄ=S+0.5 C

J₀=−0.5 C cos(2α)

J₄₅=−0.5 C sin(2α)

Correction values are values that characterize a correction means, such as a spectacle lens, for example, which is fitted to the respectively determined ametropia, and contain at least the value S for sphere in the conventional notation of sphere, cylinder, and axis. Should the ametropia contain an astigmatism, the correction values also contain the values C and a for cylinder and axis. Moreover, the correction values may comprise further values, for example prism and the base thereof for correcting strabismus (squint) and a value of addition (near addition), which corrects presbyopia. The results of the objective refraction and the subjective refraction can be used independently or in combination for determining the correction values of the prescribed spectacles (synonyms: prescription, spectacle values, correction, prescription values). If the objective or subjective refraction data obtained by means of the objective or subjective refraction are available in the sphere, cylinder, and axis notation, the objective or subjective refraction data can be used directly as correction values. Below, objective correction values denote correction values that are based on objective refraction data and subjective correction values denote those that are based on subjective refraction data.

Approaches for making statements about the subjective refraction data or the subjective correction values of the eye from the measurement of objective refraction data of the eye have been around for a long time. Here, use can be made of aberrations of lower and/or higher order, or a combination thereof.

WO 2013/058725 A1 discloses that preliminary subjective refraction data, which an optician or ophthalmologist can use as a starting point for ascertaining subjective refraction data, can be predicted from results of objective measurements of the aberrations of the eye, which characterize wavefront aberrations of the eye. Here, ascertaining the preliminary subjective refraction data from the objective measurements of the aberrations is implemented using a linear system of equations with coefficients that have scaling factors assigned thereto, where the values for the scaling factors that are suitable for the measured eye are ascertained with the aid of an algorithm. Then, the preliminary subjective refraction data are calculated by means of the system of equations and the ascertained values for the scaling factors.

WO 2008/049503 A2 describes a method for ascertaining a spectacle lens prescription, in which the spectacle lens prescription is determined from objective refraction data or a combination of objective refraction data and ascertained subjective refraction data.

U.S. Pat. No. 7,357,509 B2 discloses metrics which predict the subjective effects of a wavefront aberration of the eye. The metrics depend on the wavefront aberration or on the wavefront aberration and the neural transfer function. In particular, U.S. Pat. No. 7,357,509 B2 describes metrics which include the neural contrast sensitivity. The metrics can be used to determine predicted correction values for correcting ametropia.

US 2015/0346512 A1 likewise discloses a method for ascertaining a spectacle lens prescription, i.e., for ascertaining correction values for correcting ametropia, from measured aberrations of the eye on the basis of metrics that represent the visual quality. In US 2015/0346512 A1, a term representing an evaluation function, which takes account of the degree of astigmatism correction in the predicted spectacle lens prescriptions, is added to the metric.

However, the previously used metrics for calculating predicted correction values for correcting ametropia are limited in terms of taking account of the sensory-physiological perception of vision. Usually, only a single physiological measurement variable (the contrast sensitivity) is taken into account. Thus, for example, in the method described in U.S. Pat. No. 7,357,509 B2, the contrast sensitivity is used in part for determining the best correction values. Here, for example, the objective refraction data are determined using a simple autorefractor or wavefront aberrometer. A metric from U.S. Pat. No. 7,357,509 B2 attempts to model both the optical and neural part of the imaging in order to predict the best possible correction values from objective refraction data. However, it is known that the contrast sensitivity, in particular, is subject to very pronounced individual differences. Since the neural transfer function, in particular, is highly nonlinear and difficult to describe under certain circumstances, the obtained results may be inaccurate. Moreover, taking account of only the contrast sensitivity describes the visual faculty as a combination of physical-optical properties and sensory-physiological perceptions with insufficient accuracy if no individual CSF (contrast sensitivity function) is used.

Hence, the previous solutions for calculating predicted subjective refraction data or predicted subjective correction values from objective measurements of the aberrations of the eye using metrics are either long-winded and/or insufficiently accurate in comparison with the actual ascertainment of the subjective refraction data using a phoropter or a trial frame.

DE 10 2014 226 824 A1 describes a method for providing a learning-based diagnostics assistance model. To this end, new or extended training data, which, as a rule, represent negative case studies, are collected. Then, educational software is used to train the diagnostic system with training data representing training examples. So-called neural networks can be used to design educational software.

WO 2005/079546 A2 discloses an apparatus for ascertaining a correlation between subjective correction values of an eye to be examined and objective refraction data of the eye to be examined. A statistical model that has been obtained by means of a neural network is used for ascertaining the correlation.

### SUMMARY

It is an object of the present disclosure to provide an advantageous apparatus for ascertaining predicted subjective refraction data or predicted subjective correction values for an eye on the basis of objective refraction data, the apparatus facilitating an increase in the reliability of the calculation of the predicted subjective refraction data or the predicted subjective correction values from the objective refraction data of the eye.

It is a further object of the present disclosure to provide an advantageous computer program product having software for ascertaining predicted subjective refraction data or predicted subjective correction values of an eye to be examined on the basis of objective refraction data, the computer program product facilitating, in particular, an increase in the reliability of the calculation of the predicted subjective refraction data or the predicted subjective correction values from the objective refraction data of the eye.

The first object is achieved with an apparatus and a computer program product as disclosed herein.

The solution according to the disclosure uses methods of machine learning. By using a training data record of objective refraction data with assigned captured subjective refraction data or assigned captured subjective correction values, a nonlinear multidimensional function or a family of nonlinear multidimensional functions is ascertained by means of a model with a sufficiently large capacity and typically with a regularization matched thereto in order to avoid overfitting, with the aid of which subjective refraction data or subjective correction values are then predicted from objective refraction data.

To this end, an apparatus according to the disclosure for ascertaining predicted subjective refraction data or predicted subjective correction values of an eye to be examined on the basis of objective refraction data of the eye to be examined comprises an evaluation unit, which comprises a calculation unit that calculates the predicted subjective refraction data or predicted subjective correction values of the eye from the objective refraction data of the eye by means of a function. The apparatus according to the disclosure is distinguished by virtue of the function being a nonlinear multidimensional function or a family of nonlinear multidimensional functions, which is the result of training a regression model or classification model which has been trained on the basis of a training data record, which, for a multiplicity of subjects, in each case comprises at least ascertained objective refraction data and assigned subjective refraction data ascertained by subjective refraction or assigned subjective correction values ascertained by subjective refraction. Discrete predicted subjective refraction data or discrete predicted subjective correction values are obtained in the case of the classification model.

In particular, the subjects can be a group of people that forms a representative cross section of the population. A larger group of people subsequently allows a more accurate prediction of subjective refraction data or subjective correction values from measured objective refraction data. If the apparatus for ascertaining predicted subjective refraction data or predicted subjective correction values should only be used to correct a certain refractive error, it is advantageous to incorporate a group of people with the corresponding refractive error as subjects instead of a group of people that forms a representative cross section of the population. As a result, it is possible to reduce the number of subjects in comparison with subjects that form a representative cross section of the population, in which the refractive error is only present in a certain percentage of persons. However, the nonlinear multidimensional function or the family of nonlinear multidimensional functions then is a function specific to the refractive error, which cannot be used in general.

Particularly if the subsequent ascertainment of predicted subjective refraction data or predicted subjective correction values of an eye to be examined should be implemented in cycloplegic fashion on the basis of objective refraction data of the eye to be examined, the measurement on the subjects can likewise be implemented in cycloplegic fashion. Here, in cycloplegic fashion means that the ability of the eye to accommodate is suppressed during the measurement of the objective refraction data. However, if the number of subjects is large enough, it is also possible using the apparatus to ascertain the predicted subjective refraction data or the predicted subjective correction values in non-cycloplegic fashion, i.e., without the accommodation of the eye being suppressed, even if the measurement of the objective refraction values in the group of people forming the subjects has been carried out in cycloplegic fashion because a large number of subjects renders it possible to model the effect of the accommodation of the eye with sufficient accuracy when training the regression model or classification model. Naturally, it is also possible to undertake the measurement of the objective refraction values in the group of people forming the subjects in non-cycloplegic fashion. In particular, the nonlinear multidimensional function or the family of nonlinear multidimensional functions also supplies refraction data for children if these are measured in non-cycloplegic fashion. The refraction results obtained with the nonlinear multidimensional function or with the family of nonlinear multidimensional functions are comparable to measurements in the case of children measured under cycloplegia.

According to Enzyklopädie der Wirtschaftsinformatik—Onine-Lexikon; Editors Norbert Gronau, Jörg Becker, Karl Kurbel, Elmar Sinz, and Leena Suhl (available at the url: www.enzyklopaedie-der-wirtschaftsinformatik.de), as of Sep. 27, 2013, a classification model is a mapping that describes the assignment of elements to predetermined classes. In the present case, there is an assignment of objective refraction data as the elements or the data objects to subjective refraction data as classes, wherein objective refraction data may be provided, for example, in the form of Zernike coefficients and the respective class of subjective refraction data may be provided, for example, in the conventional notation with discrete values for sphere, cylinder, and axis. In the classification model, the classes of subjective refraction data differ from one another in at least one of the discrete values for sphere, cylinder, and axis. Instead of being defined by discrete values for sphere, cylinder, and axis, the classes of subjective refraction data may also be defined, however, by other discrete specifications that reproduce the subjective refraction, for example by discrete values of power vector coefficients. When assigning elements or data objects to predetermined classes, the class characteristic of the discrete classification variables, i.e., the discrete values for sphere, cylinder, and axis in the specified example, arises from the characteristics of the attributes of the data objects, i.e., from the characteristic of the Zernike coefficients as attributes of the objective refraction data in the specified example. The basis for a classification model is formed by a data pool, the objective and subjective refraction data measured on the subjects in the present example, the data objects of which, provided by the objective refraction data in the specified example, are each assigned to a predetermined class, i.e., respectively one class of subjective refraction data defined by the values for sphere, cylinder, and axis in the specified example. A classification model is used to predict the class to which data objects belong, the class belonging of which is unknown to date, i.e., to predict the belonging of objective refraction data measured on a patient to a class defined by the values for sphere, cylinder, and axis in the present case.

In contrast to a classification model, a regression model represents a dependent, continuous variable using a plurality of independent variables. Thus, in the specified example, there is no assignment of the objective refraction data to classes of subjective refraction data, which are defined by discrete values for sphere, cylinder, and axis, but there is in each case an individual assignment to subjective refraction data, which are defined by continuous values for sphere, cylinder, and axis. Consequently, a regression model can likewise be used to predict the unknown value of the dependent variables by the characteristics of the associated independent variables. The difference to a classification model lies in the cardinality of the dependent variables. There is a discrete variable in the case of a classification model, while a regression model has a continuous variable.

The evaluation unit can be designed, in particular, for processing objective refraction data in the form of objectively ascertained wavefront aberrations, for example in the form of coefficients of an orthogonal function system that specify objective wavefront aberrations, e.g., in the form of Zernike coefficients. Moreover, the evaluation unit can be designed to output predicted subjective refraction data or predicted subjective correction values in the form of power vector coefficients, using conventional notation with sphere, cylinder, and axis, or in any other suitable notation.

All approaches that achieve a sufficiently high performance may be used as a classification model or regression model. By way of example, suitable approaches include a linear regression on nonlinear features (polynomial features, deep network features, etc.), neural networks, i.e., networks made of artificial neurons, with a plurality of layers, support vector regression with nonlinear kernels (e.g., a radial basis function kernel), decision trees, Gaussian processes, ensembling with a plurality of regressors, etc. A reformulation of the regression model as a classification model—by discretizing the output values such as the sphere values, for example—supplies an equivalent solution. Derived therefrom, all classification methods that achieve a sufficiently high performance are suitable. Examples of conceivable classification methods include: neural networks, support vector machines, nearest neighbors, linear/quadratic discriminant analysis, naïve Bayes, decision trees, Gaussian processes, ensembling a plurality of classifiers, etc. With a sufficient capacity, it is possible to capture the mapping (objective refraction→subjective refraction; both in optical and neural fashion) without the mechanical model for the optical and neural part having to be known.

Ascertaining predicted subjective refraction data or predicted subjective correction values can be implemented very quickly using the apparatus according to the disclosure because there is no need to measure the contrast sensitivity. Moreover, the apparatus according to the disclosure facilitates the reliable prediction of subjective refraction data or subjective correction values for the patient from objective refraction data of the patient using the nonlinear multidimensional function resulting from the trained regression model or classification model or using the family of nonlinear multidimensional functions resulting from the trained regression model or classification model.

The prediction of subjective refraction data or predicted subjective correction values includes, inter alia, the neural transfer function that, as mentioned at the outset, is highly nonlinear. Therefore, results obtained using the contrast sensitivity are inaccurate; this can be traced back to the nonlinearity of the neural transfer function. By contrast, the use of a nonlinear multidimensional function allows a reliable and accurate calculation of predicted subjective refraction data or predicted subjective correction values. Here, the trained model, i.e., the nonlinear multidimensional function or family of nonlinear multidimensional functions resulting therefrom, captures all optical and neural contributions that are reproducible over the measurement population, i.e., the group of people forming the subjects, represented by the training data records. As a result of use being made of a nonlinear function or a family of nonlinear functions, the highly nonlinear neural transfer function can be taken into account particularly well when calculating the subjective refraction data or subjective correction values.

The multidimensional property of the nonlinear function or of the family of nonlinear functions moreover allows a plurality of optical aberrations to be taken into account over a plurality of variables when calculating the subjective refraction values or the subjective correction values from the objective refraction values. The more variables are present, i.e., the higher the dimension of the nonlinear function or of the family of nonlinear functions, the more aberrations can be taken into account. If the objective refraction data are provided in the form of Zernike coefficients, for example, the dimension corresponds to the number of Zernike coefficients taken into account. To calculate sufficiently accurate subjective correction values from the objective refraction data, at least three variables, i.e., for example, at least three Zernike coefficients, should be present; i.e., at least a three-dimensional nonlinear function or family of three-dimensional nonlinear functions should be present. However, typically, at least ten variables, i.e., for example, at least ten Zernike coefficients, should be present; i.e., at least a ten-dimensional nonlinear function or family of ten-dimensional nonlinear functions should be present. It is particularly advantageous if the number of variables, i.e., for example, the number of employed Zernike coefficients, lies between 30 and 50 because very accurate modeling of the optical aberrations is possible in that case.

As a result of the improved consideration of physical-optical properties and sensory-physiological perceptions for the sense of vision made available by the apparatus according to the disclosure, it is moreover possible to predict specific correction values for different visual requirements, such as for scotopic or mesopic vision, for example. By way of example, this can be implemented by virtue of training data records being used when training the regression modules, in which training data records objective refraction data for a larger pupil are present and the subjective refraction data ascertained by the subjective refraction or the subjective correction values ascertained by the subjective refraction contain those subjective refraction data or subjective correction values that are required for a specific correction (e.g., night conditions). To this end, the evaluation device can also take account of, in particular, pupil diameter data of the eye to be examined.

The evaluation device comprises the regression model or classification model in an exemplary embodiment. Moreover, it comprises a training module, by means of which the regression model or classification model is trainable on the basis of a training data record, which, for a multiplicity of subjects, in each case comprises ascertained objective refraction data and assigned subjective refraction data ascertained by subjective refraction or assigned subjective correction values ascertained by subjective refraction, for the purposes of obtaining the nonlinear multidimensional function or the family of multidimensional functions. In particular, this can further optimize the multidimensional function or the family of multidimensional functions or adapt these to specific requirements where necessary if the evaluation device comprises an input interface, connected to the training module, for receiving or entering training data records. Then, further training of the regression model can be implemented using the received training data records.

In a further exemplary embodiment, the evaluation device comprises a regularization unit that is matched to the regression model or classification model in order to avoid overfitting when training. Overfitting would lead to the nonlinear multidimensional function or the family of nonlinear multidimensional functions being fitted to statistical variations rather than the underlying statistical relationship between the objective refraction data and the subjective refraction data ascertained by the subjective refraction or the subjective correction values ascertained by the subjective refraction. To avoid this, the regularization unit performs a regularization; i.e., it introduces additional information into the training by means of which fitting of the nonlinear multidimensional function or the family of nonlinear multidimensional functions to statistical variations is suppressed.

In addition to the evaluation device, the apparatus according to the disclosure may also comprise a refraction measuring apparatus for determining and providing the objective refraction data of the eye to be examined. Here, the refraction measuring apparatus may comprise an output interface for outputting the objective refraction data, the output interface being connected to a corresponding input interface of the evaluation unit for receiving the objective refraction data. For the purposes of determining the objective refraction data, the refraction measuring apparatus can be based on methods such as autorefraction, photorefraction, and on wavefront aberrometers or other objective refractors.

Within the scope of the apparatus according to the disclosure, the nonlinear multidimensional function or the family of nonlinear multidimensional functions also can be configured to calculate the predicted subjective refraction data or predicted subjective correction values of the eye from the objective refraction data of the eye and the pupil diameter of the eye, wherein the training data record used for training purposes in each case also comprises a captured pupil diameter for each subject. It is also an option in this case for the apparatus according to the disclosure to comprise a pupil diameter measuring apparatus for determining the pupil diameter of the eye to be examined. Here, the pupil diameter measuring apparatus can be embodied to create pupil diameter data that represent a measured pupil diameter and can comprise an output interface for outputting the pupil diameter data, the output interface being connected to the corresponding input interface of the evaluation device for receiving the pupil diameter data.

The apparatus according to the disclosure can be integrated, in particular, in an optical observation appliance that comprises a module for measuring refraction. By way of example, such optical observation appliances can be autorefractors, aberrometers, microscopes, surgical microscopes, telescopes, binoculars, etc.

Moreover, the disclosure provides a computer program product having program code for ascertaining predicted subjective refraction data or predicted subjective correction values of an eye on the basis of objective refraction data of the eye. Here, a computer program product should be understood to mean program code that is stored on a suitable medium, in particular a non-transitory storage medium, and/or callable from a suitable medium, in particular a non-transitory storage medium. Any medium suitable for storing software, for example a DVD, a USB stick, a flash card or the like, can be used to store the program code. By way of example, the program code can be called via the Internet or an intranet or via any other suitable wireless or wired network.

The program code of the computer program product according to the disclosure is configured in such a way that the ascertainment of predicted subjective refraction data or predicted subjective correction values is implemented with the subsequent method steps when the computer program product is loaded on a computer and/or executed on a computer, specifically:
i) providing the objective refraction data;
ii) calculating the predicted subjective refraction data or the predicted subjective correction values on the basis of a nonlinear multidimensional function or a family of nonlinear multidimensional functions, which is the result of training a regression model or classification model, wherein the regression model or classification model has been trained on the basis of a training data record, which, for a multiplicity of subjects, in each case comprises at least ascertained objective refraction data and assigned subjective refraction data ascertained by subjective refraction or assigned subjective correction values ascertained by subjective refraction, and
iii) outputting the predicted subjective refraction data or predicted subjective correction values of the eye.

Here, the program code can contain the regression model or classification model and an optional method step of training the regression model or classification model on the basis of a training data record.

The program code can be designed, in particular, for receiving objective refraction data in the form of objectively ascertained wavefront aberrations, for example in the form of coefficients of an orthogonal function system that specify objective wavefront aberrations, for instance in the form of Zernike coefficients. Moreover, it can be configured to output predicted subjective refraction data or predicted subjective correction values in the form of power vector coefficients, using conventional notation with sphere, cylinder, and axis, or in any other suitable notation.

To avoid overfitting when training the regression model, the program code may moreover contain a regularization that is matched to the regression model or classification model. Expressed differently, the program code introduces additional information into the training which, during training, suppresses fitting of the nonlinear multidimensional function or of the family of nonlinear multidimensional functions to statistical variations rather than the underlying statistical relationship between the objective refraction data and the subjective refraction data ascertained by the subjective refraction or the subjective correction values ascertained by the subjective refraction.

The program code can be configured in such a way that, in the method step of calculating the predicted subjective refraction data or the predicted subjective correction values, the pupil diameter of the eye is also taken into account in addition to the objective refraction data of the eye. Then, the training data record used to train the regression model or classification model respectively also comprises a captured pupil diameter for each subject.

The computer program product allows an apparatus according to the disclosure to be realized with the aid of a commercially available computer. Therefore, the advantages of the apparatus according to the disclosure are referred to in respect of the obtainable advantages.

### BRIEF DESCRIPTION OF THE DRAWINGS

The disclosure will now be described with reference to the drawings wherein:

FIG. 1 shows an apparatus for ascertaining subjective refraction data in the form of a block diagram; and

FIG. 2 shows a schematic illustration of ascertaining predicted subjective refraction data or predicted subjective correction values of an eye to be examined on the basis of measured objective refraction data of the eye to be examined.

### DESCRIPTION OF EXEMPLARY EMBODIMENTS

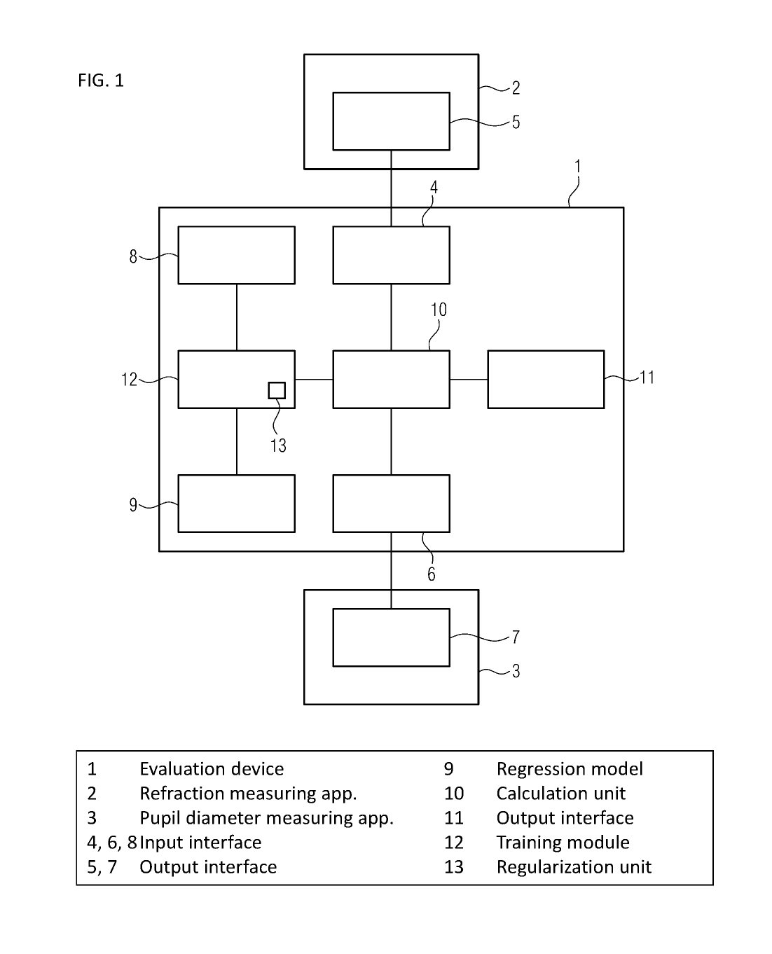

An exemplary embodiment of the apparatus according to the disclosure for ascertaining predicted subjective refraction data or predicted subjective correction values of an eye to be examined on the basis of ascertained objective refraction data of the eye to be examined is explained below with reference to FIG. 1. In particular, FIG. 1 shows an evaluation device 1, which may optionally be connected to a refraction measuring apparatus 2 and a pupil diameter measuring apparatus 3.

The evaluation device 1 comprises an input interface 4 for receiving objective refraction data, the input interface being connected to an output interface 5 of the refraction measuring apparatus 2 in the present exemplary embodiment. The refraction measuring apparatus 2 outputs objective refraction data via the output interface 5, the objective refraction data representing the objective refraction data measured on an eye. A wavefront aberrometer is used in the present exemplary embodiment for the purposes of ascertaining the objective refraction data. However, alternatively use can also be made of refraction measuring apparatuses that are based on autorefraction or photorefraction. However, instead of a refraction measuring apparatus 2, the input interface 4 can also receive the objective refraction data from a data storage, for instance a USB stick, a CD, a DVD, or a memory card. It is likewise possible to obtain the objective refraction data over a network, for example over the Internet or an intranet.

Moreover, the evaluation device 1 comprises a further input interface 6, which is connected to an output interface 7 of the pupil diameter measuring apparatus 3 which, for example, may also be integrated in the refraction measuring apparatus 2. The input interface 6 receives pupil diameter data, which are output via the output interface 7 by the pupil diameter measuring apparatus and which represent the measured pupil diameter of the eye. In this case, too, the input interface 6 can receive the pupil diameter data from a data storage, for instance a USB stick, a CD, a DVD or a memory card, rather than the pupil diameter measuring apparatus 3. It is likewise possible, again, to also obtain the pupil diameter data from a network, for example over the Internet or an intranet.

Moreover, the evaluation device 1 in the present exemplary embodiment comprises an optional further input interface 8, by means of which training data records can be input into the evaluation device 1 where necessary. The meaning of these training data records will be explained below. The training data records, too, can be obtained from a data storage, for instance a USB stick, a CD, a DVD or a memory card, or over a network, for example over the Internet or an intranet.

As further components, the evaluation device 1 in the present exemplary embodiment comprises a regression model 9, a calculation unit 10, a training module 12 for training the regression model 9, and an output interface 11. In the present exemplary embodiment, the training module 12 moreover contains a regularization unit 13 in order to avoid overfitting during the training of the regression model 9.

The calculation unit 10 is connected to the input interface for the purposes of receiving objective refraction data 4 and to the regression model 9. The predicted subjective refraction data or predicted subjective correction values are calculated in the calculation unit 10 on the basis of the received objective refraction data. Then, the calculation result is output by way of the output interface 11.

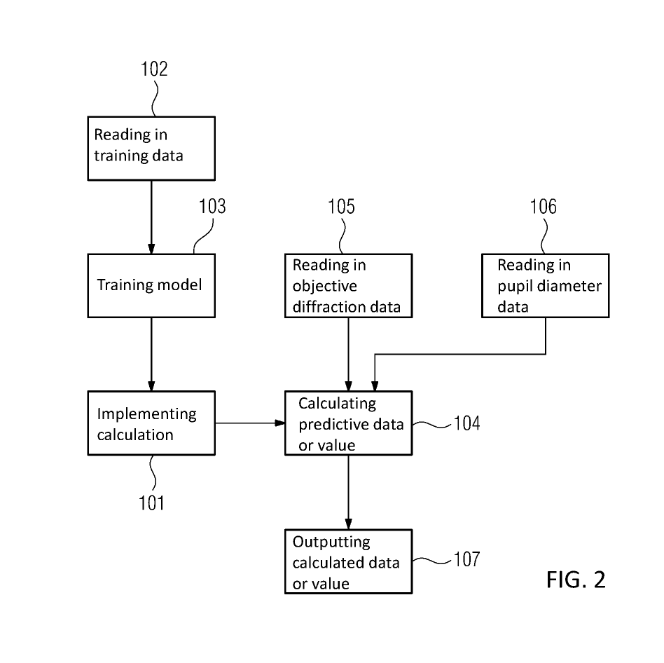

Calculating the predicted subjective refraction data or the predicted subjective correction values is described with reference to the block diagram illustrated in FIG. 2. In the present exemplary embodiment, the calculation is implemented on the basis of a nonlinear multidimensional function (block 101), which is, or has been, obtained by training the regression model 9. However, it is also possible to carry out the calculation on the basis of a family of nonlinear multidimensional functions that were obtained by training a corresponding regression model 9. For the purposes of training the regression model 9, training data are read (block 102) via the input interface for training data records 8 in the present exemplary embodiment, the training data containing objective refraction data ascertained for a measurement population and assigned subjective refraction data ascertained by subjective refraction or assigned subjective correction values ascertained by subjective refraction. Here, the ascertained objective refraction data and the subjective refraction data ascertained by subjective refraction or the subjective correction values ascertained by subjective refraction contain, for each person of the measurement population, an ascertained objective refraction data record and a subjective refraction data record assigned to this objective refraction data record and ascertained by subjective refraction and/or a subjective correction value record assigned to this objective refraction data record and ascertained by subjective refraction.

In the present exemplary embodiment, the ascertained objective refraction data are available in the form of Zernike coefficients and the ascertained subjective refraction data or the ascertained subjective correction values are available in the sphere, cylinder, and axis notation pursuant to DIN EN ISO 13666-213. Moreover, the training data in the present exemplary embodiment also contain data about the measured pupil diameter, which are assigned to the objective refraction data, for each person of the measurement population. Thus, the training data contain a number of training data records, as a rule, a large number of training data records, which each represent objective refraction data ascertained on a subject, the ascertained pupil diameter and the subjective refraction data ascertained by subjective refraction or the subjective correction values ascertained by subjective refraction.

In the present exemplary embodiment, training (block 103) is implemented with the aid of a deep neural network (DNN), with the aid of which the mean square deviation between, on the one hand, the subjective refraction data or subjective correction values predicted by the regression model on the basis of objective refraction data and, on the other hand, the subjective refraction data ascertained by subjective refraction or subjective correction values ascertained by subjective refraction for these objective refraction data is minimized. To this end, a small multi-layered feedforward network with a single linear initial unit is used in the present case. The network has two hidden layers with 128 and 37 hidden units, respectively, and parametric nonlinearities for a rectifier, as described in He, K. et al., “Delving deep into rectifiers: Surpassing human level performance on ImageNet classification”, International Conference on Computer Vision, 2015, available at url: doi.org/10.1109/ICCV.2015.123. The initial weights are obtained by a Gaussian function with a mean of 0 and a standard deviation of √(2/n), where n represents the number of incoming connections. This procedure is likewise described in He et al. In principle, developments of the network architecture, i.e., of the number of layers, number of neurons and type of the nonlinearity, are modifiable. Depending on the strength of the modifications, this may have an influence on the performance (mean squared error, cost functions, etc.). Likewise, alternative initializations to the network parameters are also conceivable. All data are pretreated by subtraction of the mean value of each feature dimension, which has been calculated by way of the training data record. By way of example, RMSprop with a learning rate of 0.001 can be used for training of the network to minimize the mean square deviation between the predicted subjective refraction data or correction values and the subjective refraction data or correction values ascertained by subjective refraction that are contained in the training data records. This procedure is described by Hinton et al. in “Neutral Networks for Machine Learning, Lecture 6,” obtainable from the url: www.cs.toronto.edu/˜tijmen/csc321/slides/lecture_slides_lec6.pdf. To avoid overfitting, small weights in the network by means of L2 regularization with lambda=0.02 are typical for the present exemplary embodiment.

As an alternative to training with a DNN, there is the possibility of carrying out the training by means of an elastic net on polynomial features. To this end, for example, a linear regression is carried out with a combination of an L1 (lasso) and an L2 (ridge) regularization (elastic net) on set basis functions. Here, the basis functions are given by a polynomial combination of the features up to and including the second order. Hyperparameters, which control the overall strength a of the regularization and the relative distribution ρ of lasso and ridge, can be set by way of a cross validation on the training data record.

As a further alternative, there is the option of carrying out the training on the basis of a support vector regression with a radial basis function kernel. In the case of the support vector aggression, a nonlinearity can be achieved by virtue of use being made of a radial basis function kernel. Then, the optimization of the hyperparameters can be carried out by means of cross validation on the training data. Suggestions for values of C and ε are contained in Kaneko et al., “Fast Optimization of Hyperparameters for Support Vector Regression Models with Highly Predictive Ability,” Chemometrics and Intelligent Laboratory Systems, 142, pages 64 to 69.

The aforementioned methods were tested by virtue of a tenfold cross validation being carried out. That is to say, for each combination of subjective refraction data or correction values and for each of the three described approaches for training, 10 models were tested on the basis of a data record of which 90% found use as training data and the remaining 10% were used to check the accuracy of the ascertainment of the predicted subjective refraction data or correction values from the objective refraction data. All three methods for machine learning and the check were implemented in Python 2.7. The deep learning library “keras” was used for the neural network part; “scikit-learn” was used for the elastic net and the support vector regression.

Thus, referring to FIG. 2 again, training data are read (block 102) and trained (block 103) using one of the aforementioned methods, DNN in the present exemplary embodiment. The result of the training then is the multidimensional nonlinear function (block 101), which is ultimately used to calculate the predicted subjective refraction data or the predicted subjective correction values from the objective refraction data (block 104).

It should be noted here that the training of the regression model for the purposes of obtaining the nonlinear multidimensional function can be carried out in advance such that this step need not be carried out again in the subsequent calculation of the predicted subjective refraction data or the predicted subjective correction values from the objective refraction data (block 104). Calculating the predicted subjective refraction data or the predicted subjective correction values then only requires reading (block 105) of the objective refraction data and, if the nonlinear multidimensional function allows the pupil diameter to be taken into account, reading of the pupil diameter data (block 106). Since the training of the regression model (block 103) only needs to be carried out once in advance, the speed at which the calculation of the predicted subjective refraction data or the predicted subjective correction values from the objective refraction data and, optionally, the pupil diameter data (block 104) can be carried out is only restricted by the speed at which the calculation can be implemented, and hence by the computational power of the apparatus.

Finally, the calculated predicted subjective refraction data or the calculated predicted subjective correction values are output (block 107). Then, a prescription for spectacles, for contact lenses, for intraocular lenses, for refractive surgery, etc., can be created on the basis of these data. It is likewise possible to undertake a correction of the refraction of the eye by surgery on the basis of the output data.

Even though training (block 103) of the regression model 9, as a rule, is implemented before the apparatus for ascertaining the predicted subjective refraction data or the predicted subjective correction values finds use in practice, it may be advantageous if there is the option of being able to further train the regression model 9. By way of example, if additional training data records are present, the basis for ascertaining the nonlinear multidimensional function can be broadened, as a result of which it is possible to further improve the reliability when ascertaining the predicted subjective refraction data or the predicted subjective correction values from the objective refraction data. Moreover, additional data records can be used, for example, to take account of regional differences or individual differences in various populations. Moreover, it is possible to extend the field of use of the apparatus. By way of example, if the original training data did not contain pupil diameter data and the trained nonlinear multidimensional function is therefore unable to take account of the pupil diameter, it is possible to carry out renewed training by way of renewed training with training data records that also contain pupil diameter data in order to adapt the apparatus to a new field of use, for example to ascertaining predicted subjective refraction data or predicted correction values in the case of scotopic or mesopic vision.

The evaluation device 1 of the apparatus for ascertaining predicted subjective refraction data or predicted subjective correction values can be realized as an appliance that is specifically created to this end. However, it is also possible to realize the evaluation unit 1 using a commercially available computer. In this case, the input and output interfaces 4, 6, 8, 11 are realized as data interfaces and the regression model 9 and the calculation unit 10 are available in the form of software modules, which can be represented by program code. Then, the program code can be stored as a computer program product on a storage medium or callable as a computer program product from a network.

The apparatus according to the disclosure for ascertaining predicted subjective refraction data or predicted subjective correction values on the basis of objective refraction values can be integrated in an optical apparatus, which measures the objective refraction values and then undertakes a correction on the basis of the calculated predicted subjective refraction data. The correction can then be implemented manually or automatically, once or repeated or continuously and, in particular, in real time. By way of example, such an apparatus can be a refractometer, a microscope, a surgical microscope, a telescope, binoculars, smartglasses, a subjective refraction unit, etc. Depending on the optical appliance in which the apparatus for ascertaining predicted subjective refraction data or predicted subjective correction values is integrated, the evaluation unit is integrated into the appliance either on its own or in combination with the refraction measuring apparatus and/or the pupil diameter measuring apparatus. By way of example, if the optical apparatus is a refractometer, only the evaluation unit and, optionally, the pupil diameter measuring apparatus are integrated into the refractometer. By contrast, if the optical unit is a surgical microscope, the refraction measuring apparatus can moreover also still be integrated into the surgical microscope. The same also applies to other optical appliances that do not contain a refraction measuring apparatus.

If the apparatus according to the disclosure is integrated into a surgical microscope, this provides the option of undertaking intraoperative objective measurements on the patient and of predicting an assessment of the operation result or the ultimately achieved refraction, for example when implanting intraocular lenses (IOLs), on the basis of the calculated predicted subjective refraction data.

In particular, the refraction can also be measured in the case of mobile or portable appliances. Here, smart phones or screening appliances should predominantly be considered. The implementation of the disclosure in the form of a computer program product, specifically in the form of an app, lends itself in the case of smartphones, it being possible to load the computer program product onto the smartphone.

The present disclosure has been described in detail on the basis of an exemplary embodiment for purposes of explanation. However, a person skilled in the art will appreciate that it is possible to depart from the exemplary embodiment. Thus, for example, coefficients that are based on other polynomials or other variables characterizing wavefront aberrations can be used instead of the Zernike coefficients for the purposes of describing the objective refraction data. By way of example, it is conceivable to use coefficients that are based on Bessel polynomials, Jacobi polynomials, Legendre polynomials, or other orthogonal polynomials. Even the mathematical description of the objective refraction data by means of splines or other polynomials is conceivable. In an analogous fashion to the alternative mathematical description of the input data, it is also possible to use alternative formulations of the regression or classification model. In particular, for a given specific model, alternative hyperparameters that lead to equivalent solutions are often also possible within certain bounds. Therefore, the present disclosure is not intended to be restricted to the described exemplary embodiment, but rather only by the appended claims.

The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

All publications, patents and patent applications cited in this specification are herein incorporated by reference, and for any and all purposes, as if each individual publication, patent or patent application were specifically and individually indicated to be incorporated by reference. In the case of inconsistencies, the present disclosure will prevail.

### LIST OF REFERENCE SIGNS

1 Evaluation device
2 Refraction measuring apparatus
3 Pupil diameter measuring apparatus
4 Input interface
5 Output interface
6 Input interface
7 Output interface
8 Input interface
9 Regression model
10 Calculation unit
11 Output interface
12 Training module
13 Regularization unit
101 Nonlinear multidimensional function
102 Reading training data records
103 Training the regression module
104 Calculating the predicted subjective refraction data or the predicted subjective correction values
105 Reading objective refraction data
106 Reading pupil diameter data
107 Outputting the predicted subjective refraction data or the predicted subjective correction values

1. An apparatus for ascertaining predicted subjective refraction data or predicted subjective correction values of an eye to be examined on a basis of objective refraction data of the eye to be examined, the apparatus comprising:
an evaluation device including a calculation unit configured to calculate the predicted subjective refraction data or the predicted subjective correction values of the eye with a function of the objective refraction data of the eye,
wherein the function is a result of training a regression model or a classification model,
wherein the regression model or the classification model has been trained on a basis of a training data record, which, for a multiplicity of subjects, in each case includes at least ascertained objective refraction data and assigned subjective refraction data ascertained by subjective refraction or assigned subjective correction values ascertained by subjective refraction, respectively, and
wherein the function is a nonlinear multidimensional function or a family of nonlinear multidimensional functions.

2. The apparatus as claimed in claim 1, wherein the nonlinear multidimensional function or the family of nonlinear multidimensional functions is at least one three-dimensional nonlinear function or a family of three-dimensional nonlinear functions, respectively.

3. The apparatus as claimed in claim 2, wherein the nonlinear multidimensional function or the family of nonlinear multidimensional functions is at least one ten-dimensional nonlinear function or a family of ten-dimensional nonlinear functions, respectively.

4. The apparatus as claimed in claim 1, wherein the regression model or the classification model further comprises:
a training module configured to be trained on the basis of a training data record,
wherein the training data record contains, for a multiplicity of subjects, in each case ascertained objective refraction data and assigned subjective refraction data ascertained by subjective refraction or assigned subjective correction values ascertained by subjective refraction, and
wherein the regression model or the classification model is configured to obtain the nonlinear multidimensional function or the family of nonlinear multidimensional functions.

5. The apparatus as claimed in claim 4, wherein the training module further comprises:
a regularization unit that is matched to the regression model or the classification model.

6. The apparatus as claimed in claim 4, wherein the apparatus further comprises:
an input interface connected to the training module, the input interface being configured to receive or enter the training data record.

7. The apparatus as claimed in claim 1, further comprising:
a refraction measuring apparatus configured to determine the objective refraction data of the eye to be examined and to provide the objective refraction data to the evaluation device.

8. The apparatus as claimed in claim 1, wherein the nonlinear multidimensional function or the family of nonlinear multidimensional functions is configured to calculate the predicted subjective refraction data or the predicted subjective correction values of the eye from the objective refraction data of the eye and a pupil diameter of the eye, and
wherein the training data record further includes a captured pupil diameter for each subject.

9. The apparatus as claimed in claim 8, further comprising:
a pupil diameter measuring apparatus configured to determine the pupil diameter of the eye to be examined.

10. An optical observation appliance having an apparatus as claimed in claim 1.

11. A computer program product stored on a non-transitory storage medium and having program code for ascertaining predicted subjective refraction data or predicted subjective correction values of an eye on a basis of objective refraction data of the eye with a method that is performed when the program code is loaded onto a computer, executed on the computer, or loaded onto and executed on the computer, the method comprising:
providing the objective refraction data;
calculating the predicted subjective refraction data or the predicted subjective correction value on a basis of a nonlinear multidimensional function or a family of nonlinear multidimensional functions, which result from a training of a regression model or a classification model,
wherein the regression model or the classification model has been trained on the basis of a training data record, which, for a multiplicity of subjects, in each case comprises at least ascertained objective refraction data and assigned subjective refraction data ascertained by subjective refraction or assigned subjective correction values ascertained by subjective refraction; and
outputting the predicted subjective refraction data or the predicted subjective correction values of the eye.

12. The computer program product as claimed in claim 11, wherein the method further comprises:
training the regression model or the classification model on the basis of the training data record.

13. The computer program product as claimed in claim 12, wherein the method further comprises:
carrying out a regularization during the training, the regularization being matched to the regression model or the classification model.

14. The computer program product as claimed in claim 11, wherein the method further comprises:
capturing a pupil diameter for each subject,
wherein the program code for calculating the predicted subjective refraction data or the predicted subjective correction values calculates the predicted subjective refraction data or the predicted subjective correction values on a basis of a nonlinear multidimensional function or a family of nonlinear multidimensional functions, which also take into account the pupil diameter of the eye in addition to the objective refraction data of the eye, and
wherein the training data record in each case also includes the captured pupil diameter for each subject.

15. An apparatus for ascertaining predicted subjective refraction data or predicted subjective correction values of an eye to be examined on a basis of objective refraction data of the eye to be examined, the apparatus comprising:
an evaluation device including a calculation unit configured to calculate the predicted subjective refraction data or the predicted subjective correction values of the eye with a function from the objective refraction data of the eye,
wherein the function is the result of training a regression model or a classification model,
wherein the regression model or the classification model has been trained on the basis of a training data record, which, for a multiplicity of subjects, in each case comprises at least ascertained objective refraction data and assigned subjective refraction data ascertained by subjective refraction or assigned subjective correction values ascertained by subjective refraction, and
wherein the function is a nonlinear multidimensional function or a family of nonlinear multidimensional functions and the regression model or the classification model is trained with a deep neural network, an elastic net on polynomial features, or a support vector regression with radial basis function kernel.

16. A computer program product stored on a non-transitory storage medium and having program code for ascertaining predicted subjective refraction data or predicted subjective correction values of an eye on a basis of objective refraction data of the eye with a method performed when the program code is loaded onto a computer, executed on the computer, or loaded onto and executed on the computer, the method comprising:
providing the objective refraction data;
calculating the predicted subjective refraction data or the predicted subjective correction value on a basis of a nonlinear multidimensional function or a family of nonlinear multidimensional functions, which result from a training of a regression model or a classification model;
training the regression model or the classification model on the basis of a training data record, which, for a multiplicity of subjects, in each case comprises at least ascertained objective refraction data and assigned subjective refraction data ascertained by subjective refraction or assigned subjective correction values ascertained by subjective refraction,
wherein the regression model or the classification model is trained with a deep neural network, an elastic net on polynomial features, or a support vector regression with radial basis function kernel; and
outputting the predicted subjective refraction data or the predicted subjective correction values of the eye.
